(19) United States
(12) Patent Application Publication
(10) Pub. No.: US 2025/0359845 A1
(43) Pub. Date: Nov. 27, 2025

(54) ULTRASOUND MEASURING PULSER RECEIVER SYSTEMS AND METHODS

(71) Applicant: Provisio Medical, Inc., San Diego, CA (US)

(72) Inventors: Stephen Eric Ryan, San Diego, CA (US); Randy-Alexander Randolph Besprozvanny, San Diego, CA (US)

(21) Appl. No.: 18/873,565

(22) PCT Filed: Jun. 23, 2023
(86) PCT No.: PCT/US2023/026109

§ 371 Date: Dec. 10, 2024

Related U.S. Application Data
(60) Provisional application No. 63/367,266, filed on Jun. 29, 2022.

(51) Int. Cl. A61B 8/12; A61B 8/08; A61B 8/14

(52) U.S. Cl. CPC A61B 8/12; A61B 8/0891; A61B 8/14

(57) ABSTRACT

A system for processing acoustic signals that includes a digital signal module and an analog signal module. The digital signal module is configured to generate a digital excitation pulse for activating one or more ultrasound transducers and is configured to receive a digital return signal representing a responsive return signal from the ultrasound transducers.

### REFERENCE TO RELATED APPLICATIONS

This application claims the benefit of U.S. Provisional Application No. 63/367,266, filed Jun. 29, 2022, which is hereby incorporated by reference in its entirety.

### BACKGROUND

### Field of the Disclosure

The present disclosure relates generally to systems, methods, and devices that utilize ultrasound to gather dimensional and physiological information about structures such as fluid-filled body vessels.

### Description of the Related Art

Recent studies have illustrated that the predominate cause of endovascular treatment failure is inaccurate sizing of vessels or inadequate treatment to achieve the lumen dimensions desired over an entire stenotic lesion. An improperly selected, dimensioned, and/or positioned medical device (e.g., a stent) and/or treatment can lead to highly adverse outcomes including avoidable death. Typical techniques used for analyzing the structural features of blood vessels include angiography. However, angiography only provides limited and imprecise information about the size and morphology of blood vessels and often does not allow the physician to adequately assess the lesion prior to treatment. Thus there is a need for systems, methods, and devices to gather dimensional and physiological information about structures such as fluid-filled body vessels.

### SUMMARY

Embodiments of the present disclosure include novel implementations of ultrasound techniques and systems that can be used with imaging probes to approximate the dimensions and shapes of fluid-filled structures, including small-sized structures such as blood vessels.

An ultrasound measurement system is configured to obtain responsive ultrasound signals from structures (e.g., vessel walls) and blood about an imaging probe inserted into a blood vessel. Embodiments utilize a technique of obtaining the responsive signals from transducer(s) arranged about the probe to measure distances between the transducer(s) and the vessel wall. The return signals represent the echoing caused by a change in acoustic impedance along the path of the acoustic signals. In some embodiments, the time of travel of the acoustic signals is measured and used to estimate distances between the transducer and the vessel wall. Multiple such distance calculations from signals of multiple transducers about a probe may be used to generate a map of points and interconnect them (e.g., by interpolation) to represent the shape and size of the structure (e.g., such as described in U.S. Pat. No. 10,231,701 entitled “Distance, diameter and area determining device,” the entire contents of which is herein incorporated by reference).

In some IVUS systems each ultrasound transducer of an array of transducers includes a piezoelectric layer with dimensions tailored to resonate at frequencies for detecting physiological properties (e.g., occlusion, calcification, etc.) of a blood vessel. Added to the piezoelectric layer is typically a matching layer providing the transducer with an acoustic impedance interface tailored (“matched”) to efficiently transmit the acoustic energy of ultrasound waves by gradually transitioning the acoustic impedances from the piezoelectric layer to tissue that is being imaged.

Arrangements of the transducers and signal carriers (e.g., wires, cables) require balancing the needs to keep the catheter profile low for navigating extremely narrow lumens (e.g., coronary vessels) and ability to acquire ample data from the areas being analyzed. The more transducers that are used in a catheter, the more space is required to accommodate their related components. In order to activate a transducer, an initial energizing electrical pulse is delivered to the transducer(s) for them to activate at their resonant ultrasonic frequency, after which responsive acoustic return (e.g., echo) signals are received at the transducer(s). Returned signals are converted/carried as electrical signals to a signal processing/computing device for processing. The activating initial pulse used for a transducer is typically much greater in magnitude than a returning echo signal (which may be from a relatively non-distinct object or medium). The inventors have learned it is important that initial pulses do not unduly interfere with returning pulses in order to obtain sufficient data for making an accurate analysis of targeted structure/media.

The more transducers that are used, the more information and detail can be obtained through their use. However, the potential for crosstalk/noise between signals associated with activating the transducer(s) and return signals increases. Increasing the number of transducers also increases the footprint and complexity of an ultrasound probe system. For example, in order to prevent the undesired mixing/interference between signals of numerous transducers and signal carriers in close proximity, effective isolation and signal processing (e.g., isolating/filtering signals) is needed.

In some embodiments, the transducer transmits ultrasound pulses directly toward a structure and receives echo pulses returning along the same path (and/or from an adjacent transducer). To generate a pulse, the transducer must be excited with an electrical signal that causes it to resonate. In some embodiments, the proximity of targeted structures/media (e.g., vessel walls, blood) causes the timing between the pulse and return signal to be extremely short. The energy and reverberation caused by the initial pulse throughout the system can thus interfere in effectively capturing and distinguishing the return signal.

In some embodiments, a system for generating ultrasound signals and receiving return signals includes a digital signal module and an analog signal module configured to be connected to an acoustic probe. The digital signal module is configured to generate and receive digital signals that are converted to and from analog signals communicated to and from an analog signal module and acoustic transducer probe (e.g., using a digital-to-analog converter (DAC) and analog-to-digital converter (ADC), accordingly). The outgoing digital signals may represent pulses for activating a transducer (e.g., representing a particular pulse width, amplitude, and/or frequency) which are then converted into analog signals by the DAC and processed by the analog signal module before being delivered to an ultrasound transducer (e.g., in an ultrasound probe). Responsive acoustic return signals (e.g., from a structure/medium like a vessel wall) are received at the transducer and a corresponding electronic signal generated by the transducer is returned to the analog signal module.

In some embodiments, the return signal from a transducer is received through the same connection used to deliver an activating pulse. This arrangement can save significant space within a probe (e.g., a catheter probe for small blood vessels), particularly compared to an arrangement in which separate input and output connections (e.g., wires, connectors) would be used for each of multiple transducers (e.g., a transducer array). However, such an arrangement of combined input/output connections may lead to interference of a return signal by the outgoing pulse signal.

In some embodiments, a DAC converted analog pulse signal is received by the analog module, after which it is amplified by a pre-amplifier. After being amplified by the pre-amplifier, the signal is directed through an absorption diode, which is configured to direct flow from the pre-amplifier to a first split input/output of a combiner/coupler. The combiner/coupler directs outgoing flow from the absorption diode to a (impedance) matching network from which the outgoing flow is directed from the analog signal module to an acoustic transducer (e.g., in an acoustic probe). When the electronic signal pulse reaches the transducer, the transducer activates and emits an acoustic pulse. In some embodiments, a shared output/input of the analog signal module is used to transmit/receive electronic signals to/from the transducer.

In some embodiments, a switch is connected to a second of a split input/output of the coupler/combiner. In one switch state, the switch directs signals from the coupler/combiner to a sink that diverts returning signals from further processing by the analog signal module and digital signal module. In another switch state, the switch directs returning signals to a limiter that is configured to filter/attenuate/block signals that exceed a particular amplitude (e.g., signals that may result directly from the initial pulse). Returning signals passing through the limiter are directed to an output of the analog signal module and to an ADC and input of the digital signal module. In some embodiments, the analog signals are amplified by an amplifier prior to processing by the ADC.

The digital signal module receives returning signals from the analog signal module/ADC and processes the signals to ready them for analysis, display, probe guidance, and/or use for diagnosis/treatment. The digital signal module may, for example, apply filtering/enhancement and/or other processing to the signals to improve their representation of targeted structures/media (e.g., lumen wall, blood). The signals may be correlated with timing data (e.g., using a timing circuit) obtained in connection with the delivery of pulses and receipt of corresponding return signals (e.g., time of flight (TOF) data). The timing data may be used, for example, to determine the distance of travel of the signals to and from the structures/media they represent.

In some embodiments and as disclosed herein, a system for processing ultrasound signals includes a controller module including one or more processors and configured to transmit control signals to an analog ultrasound signal module, the analog ultrasound signal module including a common input/output port configured to transmit and receive ultrasound signals to and from an ultrasound transducer; a return signal output port configured to transmit returned ultrasound signals to the controller module, the returned ultrasound signals received from the ultrasound transducer through the common input/output port; a first amplifier configured to receive transducer excitation signals and to amplify the transducer excitations signal in response to receiving a first control signal from the controller module; a combiner including first and second combiner input terminals and a combiner output terminal, the combiner output terminal arranged to transmit and receive ultrasound signals to and from the common input/output port of the analog ultrasound signal module, the first combiner input terminal configured to receive amplified ultrasound excitation signals from the first amplifier and direct the excitation signals to said common input/output port; and a first switch having an input configured to receive ultrasound signals from the second combiner input terminal, the first switch configured with an output and a first state that directs received ultrasound signals to a termination and configured with a second state that directs received ultrasound signals to the return signal output port, the first switch configured to change between the first state and the second state in response to receiving a second control signal from the controller module.

In some embodiments, the controller module is configured to generate the first control signal and second control signal each including pulses of the same frequency, where the pulses of the first control signal are timed to occur within the same pulse intervals as the pulses of the second control signal. In some embodiments, the pulses of the first and second control signals have a period of about 16 microseconds or less. In some embodiments, the pulse intervals of the second control signal comprise a pulse width of about 128 nanoseconds or less. In some embodiments, the pulse width of pulses of the second control signal is longer than a pulse width of pulses of the first control signal. In some embodiments, the controller module is configured to generate a plurality of bursts of the pulses for completing one cycle of transmitting and receiving ultrasound transducer signals for a first transducer prior to beginning a second cycle of transmitting and receiving ultrasound transducer signals for a second transducer. In some embodiments, the one cycle comprises about 6 bursts of 64 pulses. In some embodiments, the analog ultrasound signal module includes an absorption diode arranged and configured to direct the analog excitation pulse from the first amplifier to the first input terminal of the combiner and configured to block return signals from the combiner from reaching the first amplifier. In some embodiments, the absorption diode includes an anti-parallel diode.

In some embodiments, the analog ultrasound signal module further includes a transducer return signal subcircuit connected between the first switch and the return signal output port, the transducer return signal subcircuit including first subcircuit and second subcircuit switches connected in series between the first switch and the return signal output; a filter configured to permit signals of a predetermined frequency, a limiter configured to limit noise signals through the filter to a predetermined amplitude, and return signal amplifier configured to amplify signals from the limiter, where the filter, limiter, and return signal amplifier are connected in series between the first and second subcircuit switches, where the first and second subcircuit switches have a first state configured to direct transducer return signals through the filter, limiter, and return signal amplifier and have a second state configured to direct transducer return signals to the return signal output while bypassing the filter, limiter, and return signal amplifier, and where the controller module is configured to set the subcircuit switches in their first state after a transducer excitation signal pulse has been transmitted from the analog ultrasound signal module and to set the subcircuit switches to their second state before any further transducer excitation signal pulse has been transmitted from the analog ultrasound signal module.

In some embodiments, the return signal subcircuit includes a third subcircuit switch connected in series between the first and second subcircuit switches and configured to direct return signals from the first subcircuit switch through the third subcircuit switch to the second subcircuit switch when the first and second subcircuit switches are set to their second state. In some embodiments, the analog ultrasound signal module includes a termination branching off of a connection between the first amplifier and the combiner, where the termination includes a termination resistor and a second switch, the second switch having a first state that directs signals passing across the termination branch to the termination and a second state that disconnects the termination. In some embodiments, the termination resistor and second switch are configured to terminate signals directed from the combiner to the first amplifier.

In some embodiments, the analog ultrasound signal module includes multiple analog channel circuits, each of the analog channel circuits includes a corresponding common input/output port, return signal output port, first amplifier, combiner, and first switch; a multi-channel switch having an input corresponding respectively to each analog channel circuit and connected to the output of the first switch of the respective analog channel circuit; and where the controller module is configured to enable or disable the transmission of transducer signals between the respective combiners of the multiple transducer channel circuits and transducers by transmitting control signals to the multi-channel switch that respectively enable or disable signal transmission across the respective inputs and output of the multi-channel switch.

In some embodiments, the controller module is programmed and configured to generate a plurality of digital excitation pulses for exciting a plurality of ultrasound transducers; convert the digital excitation pulses into analog excitation pulses; transmit the analog excitation pulses to the analog ultrasound signal module; receive a plurality of return ultrasound signals from the analog ultrasound signal module, the return ultrasound signals generated in response to the analog excitation pulses; convert the returned ultrasound signals into measurements of distances between the plurality of transducers and a structure proximate to the transducers; calculate points of a map image of the structure based on the measurements of distances; calculate curvilinear fits between the calculated points; and cause the generation of a map image of the curvilinear fits within a computerized display connected to the controller module.

In some embodiments, a method of processing ultrasound transducer signals may include receiving an excitation signal for exciting an ultrasound transducer; directing the excitation signal through an amplifier while activating the amplifier; directing the excitation signal through a first input port of a combiner and through an output port of the combiner to a transducer; receiving a return transducer signal at the output port of the combiner and directing the return transducer signal to a first switch; directing the received return transducer signal from the first switch to a termination while the amplifier is activated by the first control signal; and directing the received return transducer signal from the first switch to a return signal output while the amplifier is inactive.

In some embodiments, an analog ultrasound signal circuit includes a common input/output port configured to transmit and receive ultrasound signals to and from an ultrasound transducer; a return signal output port configured to transmit returned ultrasound signals, the returned ultrasound signals received from the ultrasound transducer through the common input/output port; a first amplifier configured to receive transducer excitation signals and to amplify the transducer excitations signal; a combiner including first and second combiner input ports and a combiner output port, the combiner output port arranged to transmit and receive ultrasound signals to and from the common input/output port of the analog ultrasound signal module, the first combiner input port configured to receive amplified ultrasound excitation signals from the first amplifier and direct the excitation signals to said common input/output port; and a first switch having an input configured to receive ultrasound signals from the second combiner input port, the first switch configured with an output and a first state that directs received ultrasound signals to a termination and configured with a second state that directs received ultrasound signals to the return signal output port, the first switch configured to change between the first state and the second state.

### BRIEF DESCRIPTION OF THE DRAWINGS

Embodiments of the disclosure will be described hereafter in detail with particular reference to the drawings. Throughout this description, like elements, in whatever embodiment described, refer to common elements wherever referred to and reference by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All figures are drawn for ease of explanation of the basic teachings of the present disclosure only; the extensions of the figures with respect to number, position, relationship and dimensions of the parts to form examples of the various embodiments will be explained or will be within the skill of the art after the present disclosure has been read and understood.

FIG. 1 is an illustrative diagram of an ultrasound catheter probe system with an array of transducers according to some embodiments.

FIG. 2A is an illustrative side perspective diagram of an ultrasound catheter probe placed within a lumen according to some embodiments.

FIG. 2B is a cross-sectional perspective diagram of the ultrasound catheter probe across lines I-I′ of FIG. 2A.

FIG. 2C is another cross-sectional perspective diagram of the ultrasound catheter probe across lines I-I′ of FIG. 2A.

FIG. 3A is an illustrative diagram of an ultrasound transducer according to some embodiments.

FIG. 3B is an illustrative time chart of signal amplitude from the ultrasound transducer of FIG. 3A according to some embodiments.

FIG. 4 is an illustrative block diagram of a system for processing signals in an ultrasound probe system according to some embodiments.

FIG. 5 is an illustrative block diagram of a signal module for processing signals in an ultrasound probe system according to some embodiments.

FIG. 6 is an illustrative block diagram of a signal module for processing signals for multiple transducers in an ultrasound probe system according to some embodiments.

FIG. 7A is an illustrative time chart of transducer and switch control signals generated in an ultrasound probe system according to some embodiments.

FIG. 7B is an illustrative time chart of transducer and switch control signals generated in an ultrasound probe system according to some embodiments.

FIG. 8 is a process flow diagram of a signal processing system for an acoustic probe according to some embodiments.

FIG. 9 is a process flow diagram of return signal processing for an acoustic probe according to some embodiments.

FIG. 10 is an illustrative time chart of a transducer activating signal and return signal according to some embodiments.

### DETAILED DESCRIPTION

Obtaining and utilizing structural information about patients is a critical aspect of diagnosing and treating many medical conditions. For example, within the field of endovascular medicine, it is important to gain structural and physiological information about diseased blood vessels when selecting among interventional techniques such as angioplasty, stents, and/or surgery. Recent studies have shown that outcomes arc significantly improved through the use of more advanced, more accurate imaging techniques.

Some imaging catheters utilize ultrasound or optical technologies. For example, Intravascular Ultrasound (IVUS) and Optical Coherence Tomography (OCT) have been used in interventional diagnostic procedures to image blood vessels to locate and characterize atherosclerosis and other vessel diseases and defects.

IVUS and OCT images can be used to determine information about a vessel, including vessel dimensions, and are typically much more detailed than the information that is obtainable from traditional angiography images, which are generally limited to two-dimensional shadow images of the vessel lumen. The information gained from more accurate imaging techniques can be used to better assess physiological conditions, select particular procedures, improve performance of the procedure and/or assess procedure outcomes.

While current IVUS and OCT systems provide additional and more detailed information compared to angiograms, these IVUS and OCT systems introduce significant additional time, cost and complexity into minimally-invasive procedures. The components of these systems (e.g., transducers, wires, imaging circuitry, fiber-optics, etc.) can occupy a large footprint within the blood vessel and must often be deployed independently and at separate times from interventional procedures (e.g., angioplasty).

There is a need for improved, reduced footprint, and more efficient, consistent acoustic imaging/measuring systems and components for obtaining information about a vessel or structure, particularly information about the diameter and multi-dimensional profile of a vessel or structure, while not sacrificing speed and accuracy for timely, efficient, and effective treatment.

In order that embodiments of the disclosure may be clearly understood and readily carried into effect, certain embodiments of the disclosure will now be described in further detail with reference to the accompanying drawings. The description of these embodiments is given by way of example only and not to limit the scope of the disclosure. It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

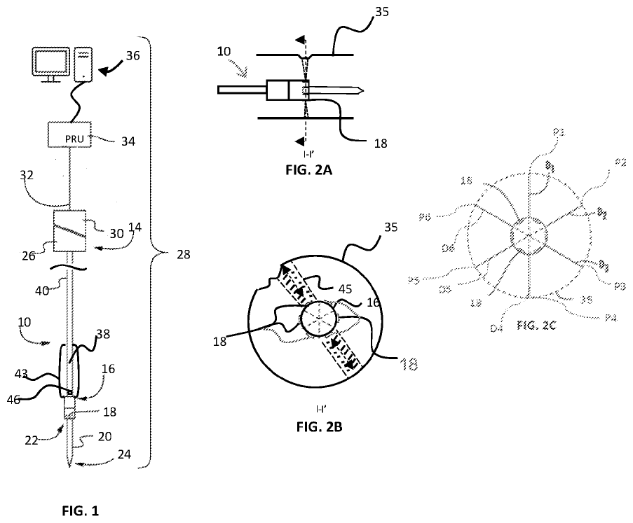

FIG. 1 is an illustrative diagram of an acoustic or ultrasound catheter probe system 28 according to some embodiments. In certain embodiments, the ultrasound catheter probe system 28 comprises an ultrasound imaging probe 10. In certain embodiments, the ultrasound imaging probe 10 includes a body 40 having a proximal end 14 and a distal end 16. In certain embodiments, the body is elongated along a longitudinal axis. In certain embodiments, the probe 10 includes a plurality of transducers 18. In certain embodiments, the probe 10 includes an elongated tip 20 having a proximal end 22 and a distal end 24. In certain embodiments, the probe 10 can include a proximal connector 26 which connects the probe 10 to other components of the system 28, including a data acquisition unit 34 and a computer system 36. In certain embodiments, the probe 10 is part of the system 28 that includes a distal connector 30, an electrical conductor 32, the data acquisition unit 34, and the computer system 36.

In some embodiments, the body 40 is tubular and includes a central lumen for containing various connectors and channels that extend toward the distal end 16. In some embodiments, the body 40 has a diameter of about 1.500 μm, 650 μm or less. These dimensions are illustrative and not intended to be limiting. In some embodiments, the diameter of the probe 10 will depend on the type of device that the probe 10 is integrated with and where the probe 10 will be used (e.g., in a blood vessel), which will become apparent to those of ordinary skill in the art in view of the present disclosure.

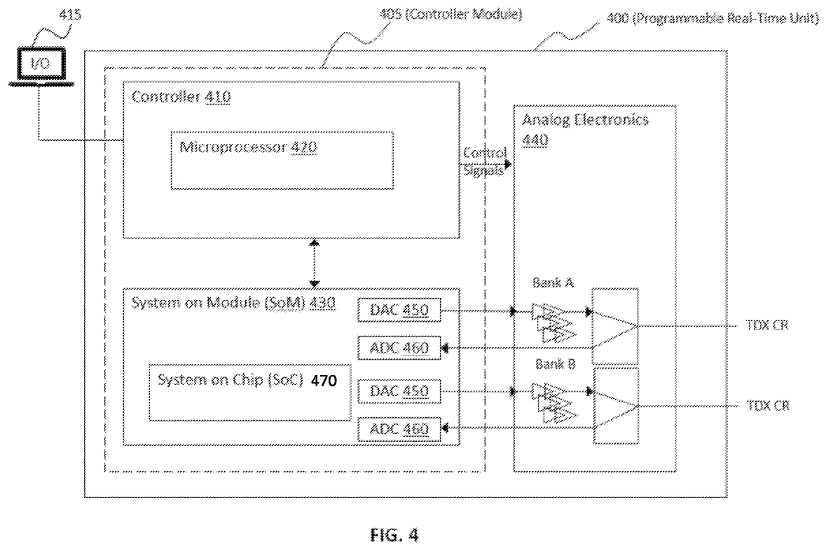

In some embodiments, the body 40 includes a portion (e.g., a ring) arranged beneath the surface of the plurality of transducers 18 to be of a hardness/stiffness so as to direct excitation/excitation vibrations and signals of the transducers 18 outwardly away from the body 40. In some embodiments, this portion is made of metal or similarly hard/inflexible material while surrounding portions of the body 40 are made of relatively more flexible material. This can mitigate excessive ringdown/feedback of transducers having fully or partially omitted backing layers (e.g., such as described with respect to transducer 300 of FIG. 3A).

In some embodiments, the proximal end 14 of the body 40 is attached to the proximal connector 26. In some embodiments, the probe 10 includes the elongated tip 20 with its proximal end 22 attached to the distal end 16 of body 40. The elongated tip 20 may be constructed with an appropriate size, strength, and flexibility to be used for guiding the probe 10 through a body lumen (e.g., a blood vessel). In certain embodiments, the elongated tip 20 and/or other components of the probe 10 may include radio-markers (e.g., visible to angiography) for precisely guiding the catheter through a lumen and positioning the plurality of transducers 18 in the desired location. In some embodiments, the probe 10 and the distal end 16 are constructed and arranged for rapid exchange use. In certain embodiments, the member 40 and the elongated tip 20 may be made of resilient flexible biocompatible material such as is common for IVUS and intravascular catheters known to those of ordinary skill in the art.

In some embodiments, the probe 10 has a tubular body 40 with a central lumen 38. In some embodiments, the probe 10 may have lumens for use with various features not shown (guidewires, fiberoptics, saline flush lumens, electrical connectors, etc.). In some embodiments, the outer diameter of the member 40 and the elongated tip 20, if present, is substantially consistent along its length and does not exceed a predetermined amount.

The plurality of transducers 18 may be incorporated with the body 40 at the distal end 16 such as described further herein to reduce the footprint of the body 40. In certain embodiments, the plurality of transducers 18 may be connected by one or more conductors extending through the lumen 38 to the data acquisition unit 34. Signals received and processed by the data acquisition unit 34 are then processed by the computer system 36 programmed to store and analyze the signals (e.g., calculate distance measurements between the catheter and lumen wall). In some embodiments, by reducing the footprint of the body 40, the space saved may be utilized to incorporate additional features (e.g., an expandable balloon 43, balloon media lumen for expanding and deflating balloon 43, and/or an ultrasound transducer 46 similar to the plurality of transducers 18 used to measure the level of expansion of balloon 43).

In some embodiments, the plurality of transducers 18 are piezoelectric. In certain embodiments, the plurality of transducers 18 may be built using piezoelectric ceramic or crystal material; as well as piezoelectric composites of ceramic or crystal material with epoxies. In some embodiments, the plurality of transducers 18 use piezoelectric crystals composed of Pb (Mg⅓Nb⅔)O3-PbTiO3 (PMN-PT) or other types of piezoelectric materials with dimensions configured to resonate, for example, at predetermined frequencies. In some embodiments, the plurality of transducers 18 are photoacoustic transducers and/or ultrasonic sensors that use MEMS (Microelectromechanical Systems) technology, such as but not limited to PMUTs (Piezoelectric Micromachined Ultrasonic Transducers) and CMUTs (Capacitive Micromachined Ultrasonic Transducers).

In certain embodiments, the operating frequency for the plurality of transducers 18 may be in the range of about 8 to about 50 MHz or even up to about 60 MHz, depending on the dimensions and characteristics of the transducers 18 and requirements of the particular application. Generally, higher frequency of operation provides better resolution and a smaller probe 10. However, the tradeoff for this higher resolution and smaller probe 10 size may be a reduced depth of penetration into the tissue of interest and increased echoes from the blood itself (making the image more difficult to interpret). Lower frequency of operation is more suitable for imaging in larger vessels or within structures such as the chambers of the heart. Although specific frequency ranges have been given, these ranges given are illustrative and not limiting. The plurality of transducers 18 may produce and receive any frequency that leaves the transducer 18, impinges on some structure or material of interest and is reflected back to and picked up by the transducer 18.

The center resonant frequency and bandwidth of the plurality of transducers 18 is generally related to the thickness of transducer materials which generate or respond to ultrasound signals. For example, in some embodiments, the plurality of transducers 18 include a piezoelectric material such as quartz and/or lead-zirconate-titanate (PZT). A thicker layer will generally respond to a longer wavelength and lower frequency and vice versa. For example, a 50 micron thick layer of PZT will have a resonant frequency of about 40 MHz, a 65 micron thick layer will have a resonant frequency of about 30 MHz, and a 100 micron layer will have a resonant frequency of about 20 MHz. As further described herein, matching and backing layers may be included, reduced, or omitted, which affect the bandwidth and other characteristics of the plurality of transducers 18.

In some embodiments, a resonant frequency of some of the plurality of transducers 18 may be centered around 20, 25, or 30 MHz while other transducers may have a resonant frequency centered around 35, 40, 45, or 50 MHz, for example. The respective materials and dimensions of the transducer layers may be configured accordingly. In certain embodiments, some subsets of the plurality of transducers 18 may be activated at the same time while other subsets activated at a separate time. In some embodiments, an electronic switch is utilized to switch connections between different transducers 18 or subsets of transducers 18.

In some embodiments, the probe 10 is connected with an actuating mechanism that may rotate and/or longitudinally move at least some portions of the probe 10 and its plurality of transducers 18. A controlled longitudinal and/or radial movement permits the probe to obtain ultrasound readings from different perspectives within a surrounding structure, for example. In certain embodiments, positioning the probe 10 and its plurality of transducers 18 in target locations may be augmented/guided by real-time imaging feedback provided by the transducers 18 and the system 28. Relative positions of the probe 10 may be tracked and recorded during such processes (e.g., by using an encoder or other position sensing tool).

In some embodiments, the system 28 is programmed to analyze and identify characteristics of the medium (e.g., blood) between the probe 10 and the structure in order to determine where the medium ends with respect to the structure (e.g., blood vessel wall, stent).

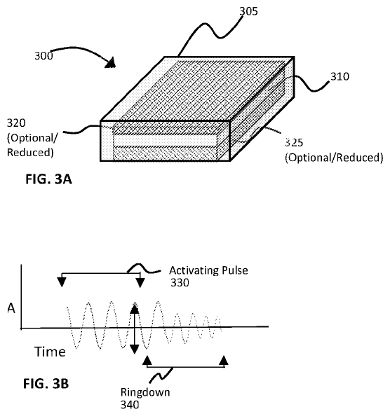

FIG. 2A is an illustrative side perspective diagram of an ultrasound catheter probe 10 placed within a lumen 35 according to some embodiments. FIG. 2B is a cross-sectional perspective diagram of the ultrasound catheter probe 10 across lines I-I′ of FIG. 2A. FIG. 2C is another cross-sectional perspective diagram of the ultrasound catheter probe across lines I-I′ of FIG. 2A. The probe 10 is shown inserted into a lumen 35. A connected computer system (i.e. 36 of FIG. 1) is programmed to cause the plurality of transducers 18 to generate one or more pulses 45 where each of the pulses 45 is incident on different portions of the lumen 35. In response to echoes from the lumen 35, the plurality of transducers 18 generate electromagnetic signals respective to the one or more pulses that reflect back from media (i.e. blood) and the lumen 35, adjacent probe 10. In certain embodiments, the electromagnetic signals are then processed by a signal processor and the computer system 36.

In certain embodiments, other pulses 45 may be similarly delivered/echoed using the other transducers 18. In some embodiments, these pulses may be delivered simultaneously or at different times. Along with identifying and associating the signals with respective transducers 18, the computer system 36 can be programmed to analyze the signals and calculate a radial distance measurement (e.g. D1, D2, . . . . D6) between each transducer 18 and structure (i.e. lumen 35 and/or stent). This may be done, for example, by utilizing time-of-flight information of the echo signals and previously determined/differentiated signatures representative of a lumen wall (e.g., of lumen 35), and a particular medium (e.g., blood) between the transducer 18 and the lumen 35.

Based on distance calculations (D1, D2, . . . . D6), the shape and dimensions of the lumen 35 may be estimated by further utilizing information including the dimensions of the probe 10 and applying interpolation and/or other mathematical fitting techniques. For example, the relative positions of points (p1, . . . p6) about lumen 35 may first be calculated and a curve fitting algorithm (e.g., spline interpolation) applied to generate a two-dimensional slice representation of the lumen 35. As described in U.S. Pat. No. 10,231,701, the entire contents of which is herein incorporated by reference, multiple slices can be calculated by taking sets of ultrasound readings along the longitudinal extent of the lumen 35 and combining them to generate a three-dimensional representation.

FIG. 3A is an illustrative diagram of an ultrasound transducer 18, 300 according to some embodiments. In certain embodiments, the transducer 18, 300 includes a piezoelectric crystal 320, a backing layer 325, a matching layer 310, and/or a protective layer 305. In certain embodiments, the piezoelectric crystal 320 is constructed to mechanically vibrate in response to ultrasonic waves incident upon the transducer 300 and, in response, generate a voltage across the crystal 320. In certain embodiments, this charge differential may be carried through a connected conductor. In certain embodiments the variance in charge across the crystal 320 may be correlated with ultrasonic frequencies incident upon the crystal over time (e.g., by the computer system 36). Similarly, in some embodiments, an electrical charge is introduced across the crystal 320 via connected conductors and an external electric power source and cause the crystal 320 to emit ultrasonic waves. Such an emission may be used to deliver ultrasound to external structures and media, after which a responsive signal (e.g., echo signals) may be monitored to detect the presence, distances, dimensions, and characteristics of those structures and media.

In certain embodiments, the backing layer 325 (aka “damping block”) is included to absorb and dampen extraneous emissions (“noise”) from the crystal 320 that are not directed toward targeted structure and media. In certain embodiments, the backing layer 325 may be substantially non-conductive so that it does not interfere with measuring signals across the crystal 320. In some embodiments, the backing layer 325 may be conductive and be utilized as an electrode. Without a backing layer 325, the noise from extraneous emissions can interfere with the detection of and accurate processing of return signals. Noise suppression from the backing layer is often needed for obtaining detailed measurements of the content and morphology of the targeted structure from the transducer 300 in a linear/scanning transducer array as employed in many IVUS systems.

In some embodiments, a reduced thickness backing layer 325 has a thickness that produces 20 dB or lower of round-trip attenuation. In some embodiments, the backing layer 325 is less than about half to about a tenth of the thickness of the crystal 320 to which it is attached. The effects of a reduced or omitted backing layer 325 may be addressed in various ways (e.g., by an expansive rigid body segment or materials layered beneath the piezoelectric layer such as further described herein).

The matching layer 310 can be used to gradually transition or better “match” the acoustic impedances between the crystal 320 and the targeted structure, thereby improving the strength and detail of return signals from the imaged structure. In some embodiments, the matching layer 310 is substantially non-conductive in order to avoid interfering with measuring charge across the crystal 320. In some embodiments, the matching layer 310 is conductive (e.g., a conductive epoxy) and can also operate as an electrode for measuring charge across the crystal 320. The protective layer 305 seals the transducer 300 from external environmental factors and media and may also be configured to dampen and/or provide “matching” characteristics similar to the matching layer 310. In some embodiments, the protective layer 305 is substantially non-conductive, or may be omitted.

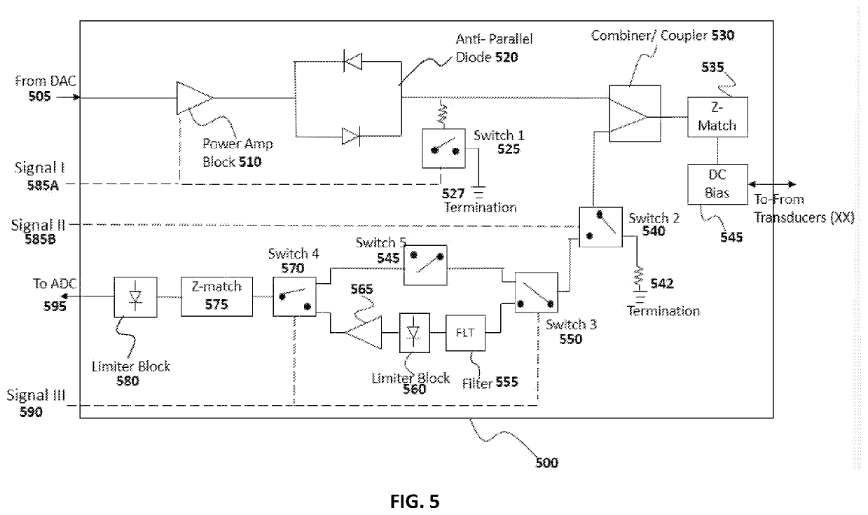

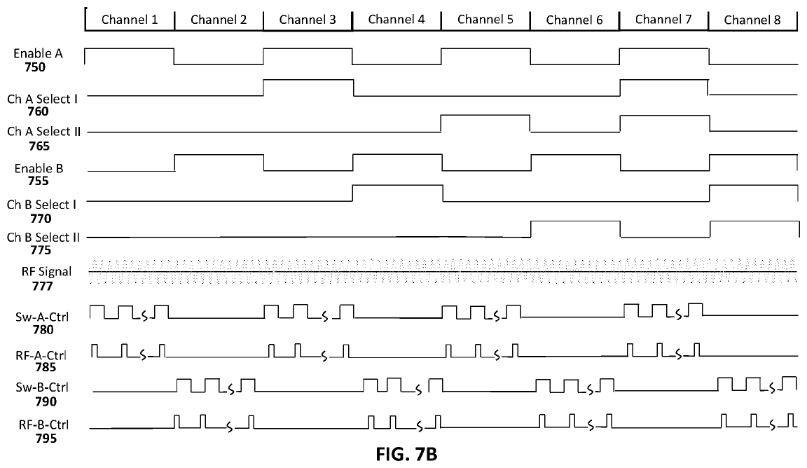

FIG. 3B is an illustrative time chart of signal amplitude from the ultrasound transducer of FIG. 3A according to some embodiments. In some embodiments, at least one transducer is utilized to both deliver and receive/detect ultrasound signals through the same electrical connections. An activating electrical pulse 330 may be delivered to a transducer and cause the transducer to emit ultrasound waves to surrounding media and structure. The activating pulse 330 may be generated such as by the systems and methods further described herein (e.g., shown in FIGS. 4-6, 8-10) and represent various types of waveforms (e.g., as shown in FIGS. 7A-7B).

During and after the delivery of the activating electrical pulses 330, the transducer(s) 300 generate return signals directly in response to the activating pulses (e.g., feedback) and in response to ultrasound received by the transducers 300 echoed from surrounding media and structure in response to the emitted pulse(s). Depending on characteristics of the transducer 300 (e.g., dampening characteristics), a certain level and period of “ringdown” 340 caused by the activating pulse 330 will result in the transducer 300 vibrating and returning ringdown feedback signals. In certain embodiments, an omitted/reduced backing layer 325 may reduce the size/footprint of the transducer 300 but may also increase the intensity/period of the ringdown period and potential interference by ringdown feedback 340 with signals representing surrounding structure. The responsive signals are fed back into the same electrical system along the same electrical connections (e.g., shown in FIGS. 4-6) through which the activating pulses 330 were generated/transmitted.

FIG. 4 is an illustrative block diagram of a system 400 for processing signals in an ultrasound probe system according to some embodiments. In certain embodiments, the system 400 includes a controller module 405 with a controller 410 with a microprocessor 420. The microprocessor 420 may be programmed and configured to initiate and control the timing of transducer excitation and processing of returning ultrasound signals. In certain embodiments, control signals include timed signals to switches and other components of an electronics module 440, which processes outgoing and incoming ultrasound transducer signals.

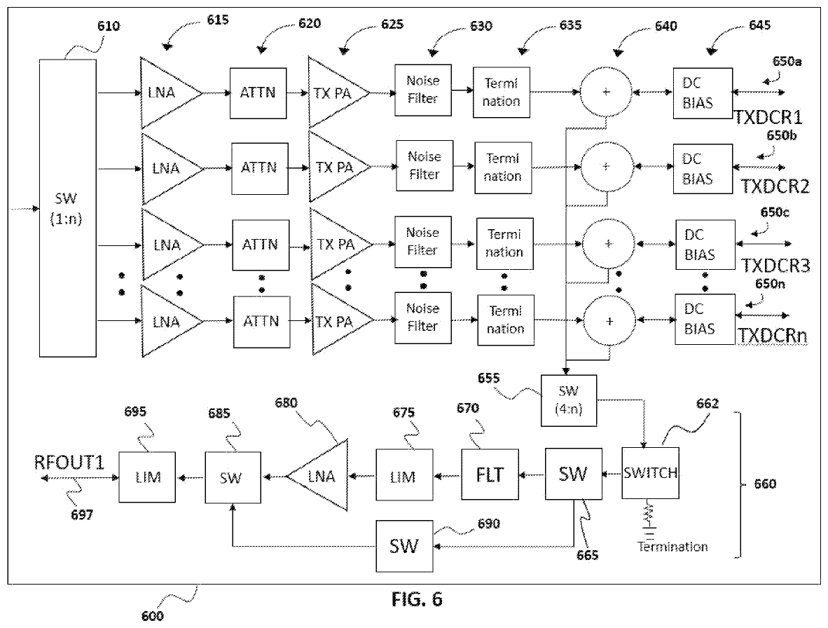

In certain embodiments, a signal processing module 430 (e.g., System on Module (SOM)) is used to process outgoing digital and incoming analog transducer signals to and from the electronics module 440. In certain embodiments, the electronics module 440 is an analog module. For processing these signals, the signal processing module 430 can include a system-on-chip (SOC) 470, digital-to-analog converter (DAC) 450, and/or analog-to-digital converter (ADC) 460. In certain embodiments, outgoing signals (e.g., through the DAC) may include electronic pulse waveforms for activating ultrasound transducers in the electronics module 440 (e.g., as further detailed in FIG. 5). In certain embodiments, the incoming signals include signals received from the same transducers after being processed by the module 440.

In certain embodiments, the electronics module 440 is configured to process outgoing and incoming ultrasound transducer signals transmitted across a common channel while substantially preventing interference with incoming transducer signals representing targeted media and/or structure. Embodiments of the electronics module 440 are described in relation to FIG. 5, for example.

In certain embodiments, one or more operator and/or communications interfaces 415 includes a display terminal/computing device for further processing and/or presenting data and/or metrics based on received transducer signals. Further processing/presentation may include graphical plots/images of targeted structures (e.g., blood vessels) and calculated measurements of the structure (e.g., diameters, areas, volumes). Communications/storage interfaces (e.g., networks/cloud/drives) may be utilized to electronically store data/calculations made of the structure. In some embodiments, an operator interface permits an operator to control and set parameters of transducer/processing operations.

FIG. 5 is an illustrative block diagram of a signal module 500 for processing signals in an ultrasound probe system according to some embodiments. In certain embodiments, the signal module 500 is an analog module. In certain embodiments, a transducer excitation signal may be received at input port 505 such as from a control module (e.g., DAC 450 of module 440 of FIG. 4). The excitation signal may represent an analog signal that matches the resonant frequency of transducers being activated. An amplification signal 585A (e.g., as shown at 730 FIG. 7A or 785 of FIG. 7B) is generated to amplify the excitation signal at an amplifier 510. In some embodiments, the amplifier 510 is configured as a low-noise amplifier a predetermined amount above a noise floor in synchronization with the signal 585A. In some embodiments, the amplifier 510 has an RF output power of at least 1 W. In some embodiments, the amplifier 510 has an output power of at least 10 W. In some embodiments, the amplifier 510 has a power output of at least 25 W. In certain embodiments, an anti-parallel/absorption diode 520 directs the amplified signal toward a combiner/coupler 530 and switch 525 while substantially preventing a feedback signal returning to the amplifier 510 and transmission electronics.

In certain embodiments, the combiner/coupler 530 is configured to direct outgoing signals to an impedance matching module 535, from which they are directed to a DC bias correction module 545, and then from signal module 500 to a transducer (e.g., in an ultrasound probe). In some embodiments, the isolation of the combiner/coupler 530 during the transmit pulse between the two coupled branches (e.g., the one input from 520 and one output to 540) is at least 6 dB. In some embodiments, the isolation is at least 20 dB. In some embodiments, the isolation is at least 30 dB. Regarding the transducer's response pulse, the coupler/combiner 530 can reduce power loss to the receiving network while maintaining high power capabilities and high isolation in a small form factor. In some embodiments, the combiner/coupler 530 can be replaced with a circulator or switch with similar isolation requirements. In some embodiments, a switch 525 is controlled/timed to direct outgoing signals to combiner/coupler 530 while timed to direct returning signals passing back through a first of the combiner/coupler 530's terminals to a termination (ground) 527 in order to prevent interference with transmission electronics (e.g., amplifier 510).

As well as being directed to the termination 542, a portion of the returning signals pass through to a second terminal of the combiner/coupler 530 to a switch 540. In certain embodiments, signals passing through the second of combiner/coupler 530's terminals are reduced in amplitude by about one-half dB. In certain embodiments, the switch 540 is controlled by a signal 585B to direct return signals to a termination during a first interval while directing return signals to a switch 550 during a second interval. In some embodiments, the first interval corresponds to when the return signals are predominantly feedback from a transducer excitation pulse and the second interval corresponds to when the feedback from the transducer excitation pulse is substantially diminished. In some embodiments, the switch 540 is controlled to direct return signals to termination 542 while the transducer excitation pulse is transmitted (e.g., when amplifier 510 amplifies transducer signals) and to direct return signals to the switch 550 when the transducer excitation pulse is not transmitted (e.g., when amplifier 510 is not amplifying transducer signals). The intervals and corresponding control signals (e.g., to switch 540 and amplifier 510) can be adjusted depending on the properties of the transducer's excitation and response (e.g., intensity and duration of ringdown).

During the second interval (e.g., when amplifier 510 is not amplifying signals) controlled by the control signal 590, first and second subcircuit switches 550 and 570 may direct return signals through a series of return signal processing components (a filter 555, a limiter block 560, and a amplifier 565) for pre-processing prior to transmission to a digital processor (e.g., controller module 405 of FIG. 4). In certain embodiments, the filter 555 suppresses signals characteristic of noise and promotes signals with peaks used to identify lumen walls. In some embodiments, the filter 555 may be a band pass filter where the low-pass component is preferably set at least Nyquist frequency (e.g., the sampling frequency is at least 2 times the transmit frequency). As an example, at 30 MHz the upper limit over which the signal would be attenuated would be 60 MHz. Thus, the high-pass component reduces any residual DC signal component and helps improve receiver network noise figure. In certain embodiments, a limiter block 560 eliminates signals below a pre-determined amplitude threshold also designed to eliminate signals representing noise. In certain embodiments, after the signals have been processed by the filter 555 and limiter block 560, they are amplified by amplifier 565 and directed through the second subcircuit switch 570.

In certain embodiments, an impedance matching module 575 and limiter block 580 match and limit the outgoing signals before being processed by an ADC 595 and digital electronics (e.g., SoM 430 of FIG. 4). The impedance matching and limiting decreases unattenuated noise/feedback from reaching the digital electronics (e.g., signals that aren't otherwise attenuated/absorbed by the combiner 530, absorption diode 520, switch blocks 540, 550, and 570, and terminations 542 and 527).

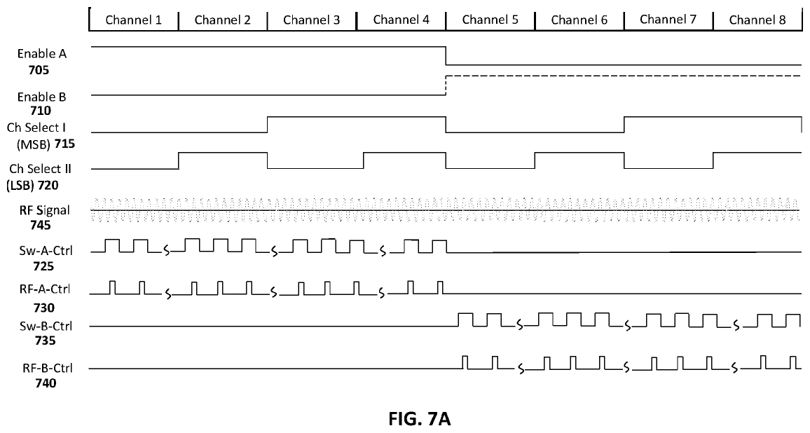

FIG. 6 is an illustrative block diagram of a signal module 600 for processing signals for multiple transducers in an ultrasound probe system according to some embodiments. In certain embodiments, the signal module 600 is analog. In some embodiments, multiple transducers arranged about a probe provide the ability to capture points about the ultrasound probe for generating a cross-sectional profile of a surrounding structure at particular times and longitudinal positions within a lumen. A 1-to-n (channel) switching block 610 is configured to switch among each of a series of components 650a, 650b . . . , 650n for directing an excitation signal to transducer channels 1 through n. These components include a low noise amplifier (LNA) 615 (e.g., amplifier block 510), an attenuation block (ATTN) 620 (e.g., absorption diode 520), a power amplifier (TX PA) 625, noise filter 630, and termination block 635 (e.g., termination 527).

In certain embodiments, a DC bias block 645 is employed at the channel inputs/outputs to ensure proper functioning of the components of the signal module 600 and to prevent distortion of incoming or outgoing signals. In certain embodiments, after signals are returned back from transducers in the ultrasound probe (not shown) and received at combiner 640, a portion of the signals are directed to the excitation transmitting circuitry (e.g., to termination 527) and a portion through a 4:n switch 655.

Depending on which channel(s) are active (e.g., designated by signals from a controller such as controller 410 to the switch 655), the switch 655 directs corresponding returned signals through return signal processing circuitry 660. Return signal processing circuitry 660 includes a shunt 662 configured to protect return signal circuitry 660 from overvoltage (e.g., excitation signal feedback). In some embodiments, first and second subcircuit switches 665 and 685 are used to direct return signals to return signal processing circuitry (e.g., a filter 670, limiter block 675, and amplifier 680) before signals exit via a limiter block 695 and output port 697 (e.g., to SOM 430). In some embodiments, the first and second subcircuit switches 665 and 685, along with a third subcircuit switch 690, are utilized to absorb and/or direct noise away from return signal processing circuitry.

In some embodiments, banks of transducer excitation circuitry, each for one or more channels (e.g., 650a, 650b, . . . , 650n), are isolated from each other in order to avoid interference between banks and channels. In some embodiments, a system includes two such banks with four channels each in order to accommodate eight channels in total (e.g., as shown in FIGS. 7A-7B). A switch (not shown) may be operated to switch between activating particular banks. In some embodiments, a system may be configured to operate multiple channels simultaneously from each of multiple banks. For example, a system may be configured to simultaneously activate transducers arranged circumferentially opposite or separated from each other on a probe (each adjacent transducer connected with a separate bank) so that the excitation signals will not significantly interfere with each other (e.g., generate crosstalk). Then, in a separate sequence, other similarly separated transducers are activated simultaneously.

FIG. 7A is an illustrative time chart of transducer and switch control signals generated in an ultrasound probe system according to some embodiments. In certain embodiments, the system includes eight channels, the first four belonging to a first bank A and a second four belonging to a second bank B (e.g., such as described above). One or more processors/timing circuits (e.g., integrated with controller 410) may be used to generate a control signal 705 for enabling transducer signals to be transmitted and received from bank A (channels (1-4)) and a second control signal 710 enables signals to and from bank B (channels (5-8)). In some embodiments, the first control signal 705 and second control signal 710 each comprise pulses of the same frequency where the pulses of the first control signal 705 are timed to occur within the same pulse intervals as the pulses of the second control signal 710. Each bank may include the processing circuitry (e.g. circuitry of signal module 600 of FIG. 6), for example as control signal 705 selects/enables each bank with one or more switches (not shown). A first switch control signal 715 and a second switch control signal 720 are configured, in combination, to activate a selection among each of the four channels in each bank. These control signals may be used with the multi-channel switching block 610 and switch 655 of FIG. 6, for example, to select among respective channels.

A transducer excitation signal 745 is generated and transmitted (e.g., through input port 505) for exciting transducers to produce ultrasound signals within structures (e.g., as shown in FIGS. 2A-2C). The excitation signal may be of a wavelength corresponding to the harmonic ultrasound wavelength of the transducer (e.g., between about 10 and 50 MHz). Control signals (e.g., signals 705 and 710) may be used (e.g., with switching block 610) to direct the excitation signal 745 to separate banks at different times. In certain embodiments, a signal 730 is generated to time an amplification of the excitation signal 745 (e.g., by signaling amplifier 510) to a level and for a period that will sufficiently excite the respective transducer for receiving ultrasound response signals.

In some embodiments, the amplifier signal 730 includes a cycle or set of pulses followed by an interval before the next sequence is applied to another channel/transducer. In some embodiments, each cycle includes 6 bursts of 64 pulses, each pulse about 128 nanoseconds or less in width and having a period of about 16 microseconds or less. The number of pulses and their widths and frequencies may be adjusted based on particular system requirements. In some embodiments, the pulse width of pulses of the second control 710 signal is longer than the pulse width of pulses of the first control signal 705.

While amplifier signal 730 is transmitted to excite a transducer, a switch control signal 725 is used to block return signals (e.g., using switch 540 of FIG. 5 to direct feedback signals to a termination 542) from being received by return signal processing circuitry. Immediately after a burst for one channel has completed (e.g., 64 pulses each over 16 microsecond cycles), the next channel is activated. Similarly, an amplifier signal 740 and a switch 735 control the return signal processing for the transducers. In some embodiments, the control switches and signals are configured to transmit and obtain signals from each of the transducers about a probe body as rapidly as possible without undue signal crosstalk or interference. This permits the probe to capture a cross-sectional map or virtual image of a surrounding structure without significant aberrations caused by movement of the probe with respect to the structure over the full period in which each of the channels/transducers are activated.

FIG. 7B is an illustrative time chart of transducer and switch control signals generated in an ultrasound probe system according to some embodiments. Instead of activating each channel/transducer in one bank before activating each channel in a second bank, control signals 750 and 755 alternate the enabling of each bank A and B, while signals 760 and 765 switch between channels within bank A and signals 770 and 775 switch between channels within bank B. For example, following an excitation of the first bank (channel 1), the first channel in the second bank (channel 4) is activated, the second channel in the first bank (channel 2) is then activated, and so on. That way, the control signals are timed to alternate (interleave) excitation of channels across the banks and thereby reduce interference between channels in the same bank.

Similar to FIG. 7A, a transducer excitation signal 777 is generated and transmitted (e.g., through input port 505) for exciting transducers to produce ultrasound signals from ultrasound probes positioned within structures (e.g., as shown in FIGS. 2A and 2B). Similar to control signals 725, 730, 735, and 740 of FIG. 7A, switch control and amplifier signals 780 and 785 for bank A, and signals 790 and 795 for bank B, respectively control the bursts of excitation pulses and return signal processing for the transducers.

In some embodiments, some channels/transducers are activated/excited simultaneously. For example, timing of the signals is configured to activate the first channel in the first bank while simultaneously activating the first channel in the second bank, and further with corresponding pairs of channels in each of the banks. In some embodiments, simultaneously activated transducers are arranged circumferentially opposite or separated from each other on a probe so that the excitation signals will not significantly interfere with each other (e.g., generate crosstalk).

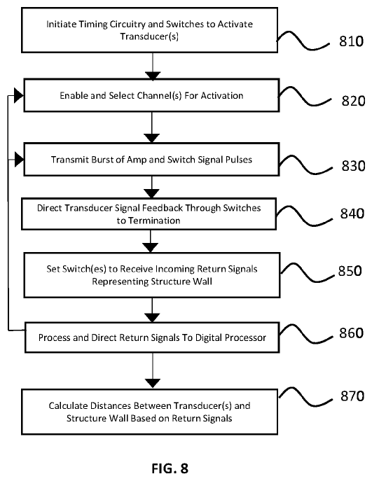

FIG. 8 is a process flow diagram of a signal processing system for an acoustic probe according to some embodiments. At block 810, timing circuitry, switches, and other system components (e.g., shown in FIG. 5) are initialized for delivering transducer excitation signals to transducers and routing feedback return signals away from return signal/measurement processing circuitry (e.g., as illustrated and described with respect to FIGS. 4-7).

At block 820, one or more transducer channels are selected for activation. Activation may include enabling one or more banks of transducer channels (e.g., banks A and B of FIG. 4) such as by using switches (e.g., switch 525) and switch signals (e.g., signals 705, 710, 750 and/or 755 of FIGS. 7A-7B). A transducer excitation signal (e.g., unamplified) is transmitted to signal processing circuitry (e.g., from a controller module DAC 450) such as at the wavelength (e.g., 30 MHz) for inducing excitation in a transducer at that particular ultrasound wavelength. At block 830, a burst of amplifier and switch pulse signals (e.g., signals 725 and 730 of FIG. 7A) are transmitted to a signal control module (e.g., signal module 500 of FIG. 5) that causes an amplified transducer excitation signal to be transmitted to one or more transducers.

At block 840, while transducers are excited, transducer feedback return signals are significantly absorbed or blocked and directed away from return signal processing circuitry such as through a termination (e.g., termination 542 of FIG. 5). At block 850, based on the changes to states of switch pulse signals and switches (e.g., the “off” state of switch 540), transducer return signals representing structures are directed through return signal processing circuitry. At block 860, the return signals are received by a digital controller (e.g., controller module 405) and digitally processed and/or stored in memory for later processing. After a cycle of transmitting a pulse or pulses of amplified transducer signals and of receiving return signals, another pulse or cycle of pulses may begin at block 830 or at block 820 utilizing a different transducer channel.

At block 870, based on the return signals received at a controller module (e.g., module 405), distances are calculated between the respective transducers and structure represented by the signal such as further referenced herein. A combination of such calculated distances may be used to determine a cross-sectional shape and metrics of the shape (e.g., diameters, area).

As further described herein, in certain embodiments, this is performed by configuring timing circuitry to time particular switches (e.g., switches 525 and 540) to divert feedback/return signals to termination/absorption points and away from the signal origination path (e.g., anti-parallel diode 520, amplifier 510) and from measurement signal processing circuitry (e.g., filter 555, amplifier 565, and ADC 595). In some embodiments, the timing circuitry is configured to generate switch-control pulses and amplifier pulses (e.g., pulses FIGS. 7A and 7B) so that the connected switches divert responsive transducer and other circuit feedback (e.g., signal amplification) at the start of transducer/feedback-response and for a short period thereafter.

In some embodiments, the switch-control pulses are timed to begin at a predetermined time before the transducer-excitation pulses and end at a predetermined time after the transducer-excitation pulse ends. In some embodiments, the predetermined time between the ends of the transducer-excitation and switch-control pulses are configured to permit the receipt of return measurement signals (e.g., of targeted structure) in measurement-processing circuitry after a substantial portion of the signal feedback has diminished.

At block 830, a switch control pulse (e.g., pulse seen in signal 725 of FIG. 7A) is transmitted to one or more switches (e.g., switch 540 of FIG. 5) which signals/sets the switches to divert return signals (e.g., feedback) to termination (e.g., termination 527) and/or divert them from being processed by signal measurement circuitry. About the same time, or shortly thereafter, an excitation pulse is transmitted to one or more transducers (e.g., pulses of signal 730 of FIG. 7A). The pulse may be generated in a digital control module (e.g., microprocessor 420/SoM 430 of FIG. 4) and processed/converted through a DAC (e.g., DAC 450) into an analog signal pulse compatible with a transducer. Before reaching a transducer, the excitation pulse is transmitted through at least a combiner/coupler (e.g., combiner/coupler 530 of FIG. 5) that is configured to significantly divert returning signals from reaching circuitry (e.g., anti-parallel diode 520) used for transmitting/generating the excitation signals.

At block 840, return signals are received through the same transducer/excitation channel from the ultrasound transducer in response to the transducer being excited/activated at block 830. Because the switches are still set to divert signals from signal processing, the return signals (including feedback) are substantially diverted to termination and away from signal processing circuitry.

At block 850, the switches are set to a state in which return signals are no longer diverted from return signal processing circuitry. In some embodiments, the change in state occurs in response to a switch control pulse ending (e.g., falling edges of pulses seen in signal of 725 in FIG. 7A). As described further herein, the change in state may be controlled to occur at a predetermined time after excitation of the connected transducer (e.g., when return feedback is substantially diminished). At block 860, the received return signals are directed to a digital processor (e.g., SoM 430) for further processing (e.g., filtering, time-of-flight/magnitude calculations).

After return signals (e.g., from media/structure) associated with the excitation of a transducer are substantially received (e.g., after a predetermined time interval), the same (or a different) transducer may be activated/excited at block 810 in order to obtain additional measurement signals. In some embodiments, a cycle of pulses of excitation and return signal processing (e.g., based on signals 730 and 725) may occur a predetermined number of times (e.g., 64 pulses at 128 ns repeated 6 times) before another channel is selected/activated. In some embodiments, the excitation/processing cycle occurs with each of a plurality of transducers arranged about a probe body within a short or substantially the same time interval (e.g., in order to obtain a cross-section of measurements absent significant intervening movement of the probe/structure).

At block 870, based on the signals received from transducers and processed at blocks 810-860, distances are calculated between the probe/transducers and surrounding structure. These distance measurements may be used to calculate cross-sectional virtual images of the structure such as referenced further herein.

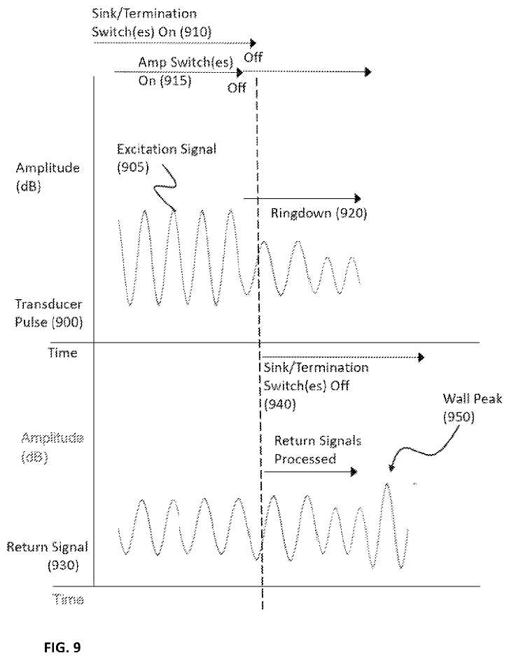

FIG. 9 is an illustrative time chart of a transducer excitation signal and return signal according to some embodiments. A transducer excitation/excitation signal is illustrated at 900. The excitation/excitation signal 900 can be generated/amplified in response to excitation pulse(s) 905 (e.g., pulses of signal 730 of FIG. 7A). A period of ringdown 920 occurs after a series of one or more excitation pulses or signals 905. During a period 910, one or more switches (e.g., switches 540 and 550 of FIG. 5) are set to divert return signals from signal processing circuitry as described further herein. During the period 910 and overlapping period 915, the excitation signal 905 is amplified (e.g., by power amplification block 510). During that period 910, a substantial portion of direct transducer excitation feedback from the amplified signals are substantially diverted from digital signal processing circuitry and do not interfere with ultrasound measurement analysis (e.g., including distance measurements of external structure).

After period 910 ends at 940, the return signal switches (e.g., switch 550) are set to a state so that return signals 930 are processed for making measurements using a digital controller (e.g., by SOM 430, controller 410 of FIG. 4). An interval is timed, in particular, so that measurement signals including those representing a wall peak at 950 are captured and processed to make a distance measurement between the receiving transducer and structure wall. Subsequently, a new cycle of transducer excitation/switch setting is performed (e.g., during a burst of pulses or for the same transducer or for a separate transducer) to make additional structure measurements.

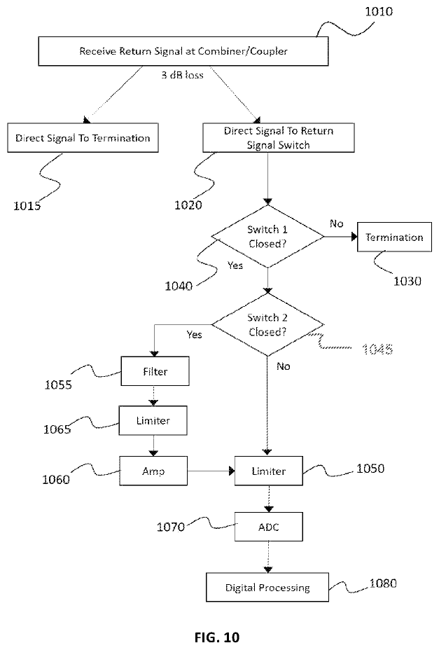

FIG. 10 is a process flow diagram of return signal processing for an acoustic probe according to some embodiments. At block 1010, a return signal in response to an excited transducer is received at processing circuitry (e.g., circuitry of signal module 500 FIG. 5). In some embodiments, the excitation signal activating the transducer is delivered through a combiner/coupler (e.g., combiner/coupler 530 of FIG. 5). The returning signal, which may include feedback from the transducer while it is excited and during a ringdown period, is directed back through the combiner/coupler and split into separate signals out through first and second input terminals of the combiner. One split of the signal is directed to a termination at block 1015 (e.g., termination 527). At block 1020, another split of the return signal is directed to a first switch (e.g., switch 540).

At block 1040, if the first return signal switch at block 1020 is in a transducer excitation state (e.g., in response to a pulse of signal 725 of FIG. 7), the return signal is directed to a termination (e.g., termination 542 of FIG. 5). The combiner/coupler will continue to receive return signals (e.g., feedback) at block 1010 and direct them to termination at block 1030 until the return signal switch state changes.

If the first return signal switch is in a state for signal response processing (e.g., after a pulse of signal 725 has ended and a substantial portion of ringdown is over), the signal is directed to a second switch (e.g., switch 550 of FIG. 5) at block 1045. In some embodiments, if the second switch is in a state for signal response processing, the signal is further directed through a return signal subcircuit 1065 (e.g., limiter block 675 of FIG. 6), a filter to reduce noise 1055 (e.g., filter 670), and/or amplifier 1060 (e.g., amplifier 680) to amplify processed return signals.

At block 1050, return signals are processed by a limiter and then converted to digital signals by an ADC at block 1070. Further received return signals may be similarly processed beginning at block 1010. At block 1080, the converted digital signals are processed such as by a digital controller (e.g., controller 410 of FIG. 4) in which processing may include storing signal data for later processing, calculating distances between transducers and surrounding structure, and/or determining the shape and various metrics pertaining to the structure such as further referenced herein.

The processes described herein (e.g., the processes of FIGS. 8 and 10) are not limited to use with the hardware shown and described herein. They may find applicability in any computing or processing environment and with any type of machine or set of machines that is capable of running a computer program. The processes described herein may be implemented in hardware, software, or a combination of the two. The processes described herein may be implemented in computer programs executed on programmable computers/machines that each includes a processor, a non-transitory machine-readable medium or other article of manufacture that is readable by the processor (including volatile and non-volatile memory and/or storage elements), at least one input device, and one or more output devices. Program code may be applied to data entered using an input device to perform any of the processes described herein and to generate output information.

The processing blocks (for example, in the processes of FIGS. 8 and 10) associated with implementing the system may be performed by one or more programmable processors executing one or more computer programs to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device, and/or a logic gate.

As described herein, generating a map image of the structure on a computer may include calculating and plotting a cross-section of the structure or a three dimensional model of the structure based on the above described processes.

The processes described herein are not limited to the specific examples described. For example, the process of FIGS. 8 and 10 are not limited to the specific processing orders illustrated. Rather, any of the processing blocks of FIGS. 8 and 10 may be re-ordered, combined or removed, performed in parallel or in serial, as necessary, to achieve the results set forth above.

Elements of different embodiments described herein may be combined to form other embodiments not specifically set forth above. Various elements, which are described in the context of a single embodiment, may also be provided separately or in any suitable sub-combination. Other embodiments not specifically described herein are also within the scope of the following claims.

1. A system for processing ultrasound signals, the system comprising:
a controller module comprising one or more processors and configured to transmit control signals to an analog ultrasound signal module, the analog ultrasound signal module comprising:
a common input/output port configured to transmit and receive the ultrasound signals to and from an ultrasound transducer;
a return signal output port configured to transmit returned ultrasound signals to the controller module, the returned ultrasound signals received from the ultrasound transducer through the common input/output port;
a first amplifier configured to receive transducer excitation signals and to amplify the transducer excitations signal in response to receiving a first control signal from the controller module;
a combiner comprising first and second combiner input terminals and a combiner output terminal, the combiner output terminal arranged to transmit and receive the ultrasound signals to and from the common input/output port of the analog ultrasound signal module, the first combiner input terminal configured to receive amplified ultrasound excitation signals from the first amplifier and direct the excitation signals to said common input/output port; and
a first switch having an input configured to receive the ultrasound signals from the second combiner input terminal, the first switch configured with an output and a first state that directs received ultrasound signals to a termination and configured with a second state that directs received ultrasound signals to the return signal output port, the first switch configured to change between the first state and the second state in response to receiving a second control signal from the controller module.

2. The system of claim 1, wherein the controller module is configured to generate the first control signal and the second control signal each comprising pulses of a same frequency, wherein the pulses of the first control signal are timed to occur within a same pulse intervals as the pulses of the second control signal.

3. The system of claim 2, wherein the pulses of the first and second control signals have a period of about 16 microseconds or less.

4. The system of claim 2, wherein the pulse intervals of the second control signal comprise a pulse width of about 128 nanoseconds or less.

5. The system of claim 4, wherein the pulse width of pulses of the second control signal is longer than a pulse width of pulses of the first control signal.

6. The system of claim 2, wherein the controller module is configured to generate a plurality of bursts of the pulses for completing one cycle of transmitting and receiving ultrasound transducer signals for a first transducer prior to beginning a second cycle of transmitting and receiving ultrasound transducer signals for a second transducer.

7. The system of claim 6, wherein the one cycle comprises about 6 bursts of 64 pulses.

8. The system of claim 2, wherein the analog ultrasound signal module comprises an absorption diode arranged and configured to direct the analog excitation pulse from the first amplifier to the first input terminal of the combiner and configured to block return signals from the combiner from reaching the first amplifier.

9. The system of claim 8, wherein the absorption diode comprises an anti-parallel diode.

10. The system of claim 1, wherein the analog ultrasound signal module further comprises:
a transducer return signal subcircuit connected between the first switch and the return signal output port, the transducer return signal subcircuit comprising:
first subcircuit and second subcircuit switches connected in series between the first switch and the return signal output;
a filter configured to permit signals of a predetermined frequency, a limiter configured to limit noise signals through the filter to a predetermined amplitude, and return signal amplifier configured to amplify signals from the limiter, wherein the filter, limiter, and return signal amplifier are connected in series between the first and second subcircuit switches;
wherein the first and second subcircuit switches have a first state configured to direct transducer return signals through the filter, limiter, and return signal amplifier and have a second state configured to direct transducer return signals to the return signal output while bypassing the filter, limiter, and return signal amplifier; and
wherein the controller module is configured to set the subcircuit switches in their first state after a transducer excitation signal pulse has been transmitted from the analog ultrasound signal module and to set the subcircuit switches to their second state before any further transducer excitation signal pulse has been transmitted from the analog ultrasound signal module.

11. The system of claim 10, wherein the return signal subcircuit comprises a third subcircuit switch connected in series between the first and second subcircuit switches and configured to direct return signals from the first subcircuit switch through the third subcircuit switch to the second subcircuit switch when the first and second subcircuit switches are set to their second state.

12. The system of claim 1, wherein the analog ultrasound signal module comprises a termination branching off of a connection between the first amplifier and the combiner, wherein the termination comprises a termination resistor and a second switch, the second switch having a first state that directs signals passing across the termination branch to the termination and a second state that disconnects the termination.

13. The system of claim 12, wherein the termination resistor and second switch are configured to terminate signals directed from the combiner to the first amplifier.

14. The system of claim 1, wherein:
the analog ultrasound signal module comprises multiple analog channel circuits, each of the analog channel circuits comprising a corresponding one of the common input/output port, return signal output port, first amplifier, combiner, and first switch; and
a multi-channel switch having an input corresponding respectively to each analog channel circuit and connected to the output of the first switch of the respective analog channel circuit,
wherein the controller module is configured to enable or disable the transmission of transducer signals between the respective combiners of the multiple transducer channel circuits and transducers by transmitting control signals to the multi-channel switch that respectively enable or disable signal transmission across the respective inputs and output of the multi-channel switch.

15. The system of claim 1, wherein the controller module is programmed and configured to:
generate a plurality of digital excitation pulses for exciting a plurality of ultrasound transducers;
convert the digital excitation pulses into analog excitation pulses;
transmit the analog excitation pulses to the analog ultrasound signal module;
receive a plurality of return ultrasound signals from the analog ultrasound signal module, the return ultrasound signals generated in response to the analog excitation pulses;
convert the returned ultrasound signals into measurements of distances between the plurality of transducers and a structure proximate to the transducers;
calculate points of a map image of the structure based on the measurements of distances;
calculate curvilinear fits between the calculated points; and
cause the generation of a map image of the curvilinear fits within a computerized display connected to the controller module.

16. A method of processing ultrasound transducer signals, the method comprising:
receiving an excitation signal for exciting an ultrasound transducer;
directing the excitation signal through an amplifier while activating the amplifier;
directing the excitation signal through a first input port of a combiner and through an output port of the combiner to a transducer;
receiving a return transducer signal at the output port of the combiner and directing the return transducer signal to a first switch;
directing the received return transducer signal from the first switch to a termination while the amplifier is activated by the first control signal; and
directing the received return transducer signal from the first switch to a return signal output while the amplifier is inactive.

17. An analog ultrasound signal circuit comprising:
a common input/output port configured to transmit and receive ultrasound signals to and from an ultrasound transducer;
a return signal output port configured to transmit returned ultrasound signals, the returned ultrasound signals received from the ultrasound transducer through the common input/output port;
a first amplifier configured to receive transducer excitation signals and to amplify the transducer excitations signal;
a combiner comprising first and second combiner input ports and a combiner output port, the combiner output port arranged to transmit and receive ultrasound signals to and from the common input/output port of the analog ultrasound signal module, the first combiner input port configured to receive amplified ultrasound excitation signals from the first amplifier and direct the excitation signals to said common input/output port; and
a first switch having an input configured to receive ultrasound signals from the second combiner input port, the first switch configured with an output and a first state that directs received ultrasound signals to a termination and configured with a second state that directs received ultrasound signals to the return signal output port, the first switch configured to change between the first state and the second state.

18. The analog ultrasound signal circuit of claim 17, further comprising a transducer return signal subcircuit connected between the first switch and the return signal output port.

19. The analog ultrasound signal circuit of claim 17, further comprising a termination branching off of a connection between the first amplifier and the combiner.

20. The analog ultrasound signal circuit of claim 19, wherein the termination comprises a termination resistor and a second switch, the second switch having a first state that directs signals passing across the termination branch to the termination and a second state that disconnects the termination.
